(19) United States
(12) Patent Application Publication
(10) Pub. No.: US 2019/0284159 A1
(43) Pub. Date: Sep. 19, 2019

(54) 4-((6-(2-(2,4-DIFLUOROPHENYL)-1,1-DIFLUORO-2-HYDROXY-3-(5-MERCAPTO-1H-1,2,4-TRIAZOL-1-YL)PROPYL)PYRIDIN-3-YL)OXY)BENZONITRILE AND PROCESSES OF PREPARATION

(71) Applicant: Dow AgroSciences LLC, Indianapolis, IN (US)

(72) Inventors: Kaitlyn Gray, Indianapolis, IN (US); Qiang Yang, Zionsville, IN (US); Sarah Ryan, Indianapolis, IN (US); Yan Hao, Zionsville, IN (US); Jim Renga, Spokane, WA (US); Nicholas R. Babij, Indianapolis, IN (US)

(21) Appl. No.: 16/462,203

(22) PCT Filed: Nov. 17, 2017
(86) PCT No.: PCT/US2017/062141

§ 371 Date: May 17, 2019

Related U.S. Application Data
(60) Provisional application No. 62/423,864, filed on Nov. 18, 2016.

(51) Int. Cl. C07D 401/06; C07D 213/65

(52) U.S. Cl. CPC C07D 401/06; C07D 213/65

(57) ABSTRACT

Provided herein is a process for the preparation of 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile.

### CROSS-REFERENCE TO RELATED APPLICATIONS

The present application claims priority under 35 U.S.C. § 119(e) to U.S. provisional patent application, U.S. Ser. No. 62/423,864, filed Nov. 18, 2016, the entire contents of which is incorporated herein by reference.

### FIELD

Provided herein is 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile and processes of preparation.

### BACKGROUND

U.S. Patent Application Ser. No. 62/163,106 describes inter alia certain metalloenzyme inhibitor compounds and their use as fungicides. The disclosure of this application is expressly incorporated by reference herein. This patent application describes various routes to generate metalloenzyme inhibiting fungicides. It may be advantageous to provide more direct and efficient methods for the preparation of metalloenzyme inhibiting fungicides and related compounds, e.g., by the use of reagents and/or chemical intermediates which provide improved time and cost efficiency.

### SUMMARY OF THE DISCLOSURE

Provided herein is the compound 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I) and processes for its preparation. In one embodiment, provided herein, is a process for the preparation of the compound of the Formula I:

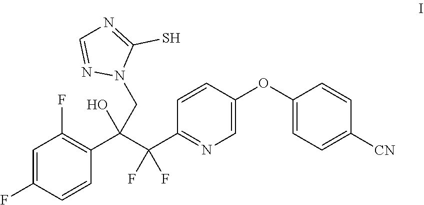 [SMILES: N#Cc1ccc(Oc2ccc(C(F)(F)C(O)(Cn3ncnc3S)c3ccc(F)cc3F)nc2)cc1]

which comprises contacting a compound of Formula II with formaldehyde or a source of formaldehyde, a thiocyanate salt, and an oxidant.

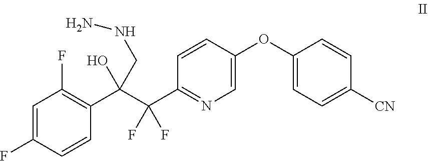 [SMILES: N#Cc1ccc(Oc2ccc(C(F)(F)C(O)(CNN)c3ccc(F)cc3F)nc2)cc1]

In another embodiment, the compound of Formula II may be prepared by contacting a compound of Formula III with hydrazine.

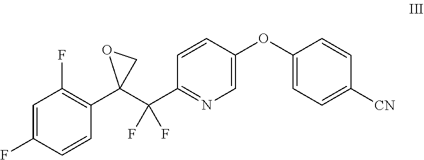 [SMILES: N#Cc1ccc(Oc2ccc(C(F)(F)C3(c4ccc(F)cc4F)CO3)nc2)cc1]

Another aspect of the present disclosure are the novel intermediates produced in the present process, viz., the compounds consisting of:

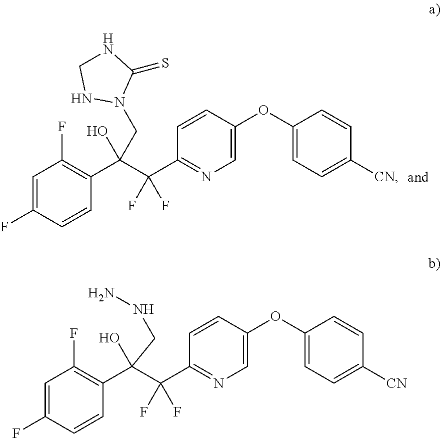 [SMILES: N#Cc1ccc(Oc2ccc(C(F)(F)C(O)(CN3NCNC3=S)c3ccc(F)cc3F)nc2)cc1 ; N#Cc1ccc(Oc2ccc(C(F)(F)C(O)(CNN)c3ccc(F)cc3F)nc2)cc1]

The term “halogen” or “halo” refers to one or more halogen atoms, defined as F, Cl, Br, and I.

The term “organometallic” refers to an organic compound containing a metal, especially a compound in which a metal atom is bonded directly to a carbon atom.

Room temperature (RT) is defined herein as about 20° C. to about 25° C.

Throughout the disclosure, references to the compounds of Formula I-III (including IIa) are read as also including optical isomers and salts. Specifically, when compounds of Formula I-III (including IIa) contain a chiral carbon, it is understood that such compounds include optical isomers and racemates thereof. Exemplary salts may include: hydrochloride salts, hydrobromide salts, hydroiodide salts, and the like.

Certain compounds disclosed in this document can exist as one or more isomers. It will be appreciated by those skilled in the art that one isomer may be more active than the others. The structures disclosed in the present disclosure are drawn in only one geometric form for clarity, but are intended to represent all geometric and tautomeric forms of the molecule. For example, the chemical structures of Formulas I and Ia are tautomeric forms of the same molecule.

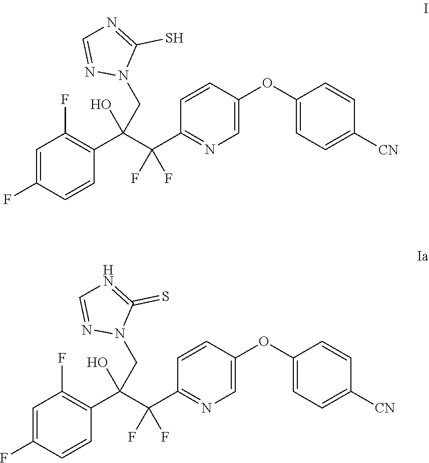

The embodiments described above are intended merely to be exemplary, and those skilled in the art will recognize, or will be able to ascertain using no more than routine experimentation, numerous equivalents of specific processes, materials and procedures. All such equivalents are considered to be within the scope of the invention and are encompassed by the appended claims.

### DETAILED DESCRIPTION

4-((6-(2-(2,4-Difluorophenyl)-1,1-difluoro-2-hydroxy-3-(5-mercapto-1H-1,2,4-triazol-1-yl)propyl)pyridin-3-yl)oxy)benzonitrile (I) is provided herein and may be prepared from 4-((6-(2-(2,4-difluorophenyl)-1,1-difluoro-3-hydrazino-2-hydroxypropyl)pyridin-3-yl)oxy)benzonitrile (II) as shown in Example 1.

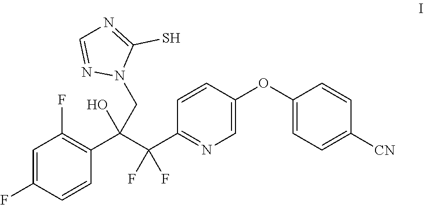 [SMILES: N#Cc1ccc(Oc2ccc(C(F)(F)C(O)(Cn3ncnc3S)c3ccc(F)cc3F)nc2)cc1]

### EXAMPLE 1

### Preparation of 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I)

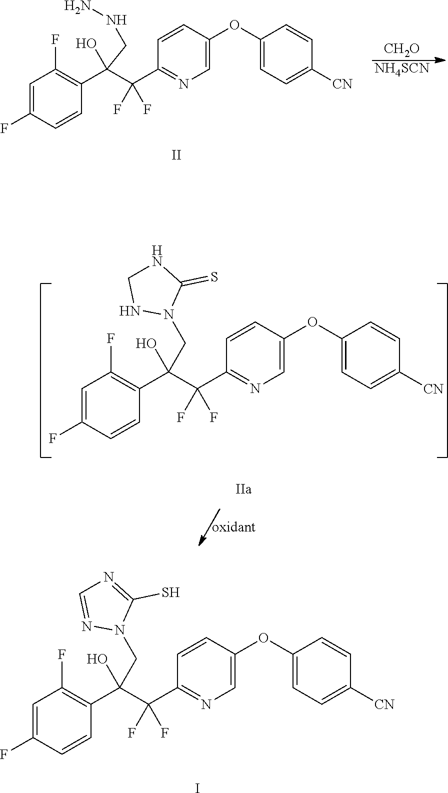

Method A: To a slurry of 4-((6-(2-(2,4-difluorophenyl)-1,1-difluoro-3-hydrazino-2-hydroxypropyl)pyridin-3-yl)oxy)benzonitrile (II) (1 g, 2.313 mmol) in ethyl acetate (9.2 mL) was added formaldehyde (37% in water, 0.172 mL, 2.313 mmol) and the mixture was stirred at room temperature for 30 min. Ammonium thiocyanate (0.194 g, 2.54 mmol) and acetic acid (0.993 mL, 17.35 mmol) were added to the reaction causing it to go from cloudy to clear. The reaction was stirred at room temperature for 1 h at which point intermediate IIa was identified by mass spec (ESIMS m/z 504.0 [(M+H)⁺]). Iron (III) chloride (10% w/v in water, 4.66 mL, 2.89 mmol) was added, and the red mixture was allowed to stir at RT overnight. The reaction was quenched with saturated sodium thiosulfate and extracted with ethyl acetate. The layers were separated, and the organic layer was dried over anhydrous sodium sulfate, filtered, and concentrated. The residue was dissolved in dichloromethane, and purified by silica gel chromatography using 0-60% ethyl acetate/hexanes as the eluent. Product containing fractions were collected and concentrated giving a white foam (580 mg). Dichloromethane (1 mL) was added to the foam and the resulting solution was stirred overnight to give a white precipitate. The solid was isolated by filtration and dried under vacuum giving 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I) (420 mg, 36.2% yield). ¹H NMR (400 MHz, CDCl₃) δ 8.44 (d, J=2.7 Hz, 1H), 7.72-7.65 (m, 2H), 7.62 (s, 1H), 7.58 (d, J=8.6 Hz, 1H), 7.50-7.36 (m, 2H), 7.10-7.02 (m, 2H), 6.80 (ddd, J=11.5, 8.6, 2.6 Hz, 1H), 6.76-6.69 (m, 1H), 5.93 (s, 1H), 5.31-5.21 (m, 2H). ¹⁹F NMR (376 MHz, CDCl₃) δ −103.15 (ddd, J=31.2, 23.4, 9.4 Hz), −108.46 (d, J=29.1 Hz), −109.02 (d, J=23.2 Hz), −109.39 (d, J=9.2 Hz). ESIMS m/z 502.0 [(M+H)⁺].

Method B: To a slurry of 4-((6-(2-(2,4-difluorophenyl)-1,1-difluoro-3-hydrazino-2-hydroxypropyl)pyridin-3-yl)oxy)benzonitrile (II) (0.05 g, 0.116 mmol) in ethyl acetate (0.463 mL) was added formaldehyde (37% in water, 8.61 μL, 0.116 mmol). The slurry was stirred at room temperature for 30 min then potassium thiocyanate (0.011 g, 0.116 mmol), sodium hydrogen sulfate (0.028 g, 0.231 mmol), and water (0.04 mL) were added. The reaction was stirred at room temperature for 2 h then was partitioned between ethyl acetate and water. The organic layer was concentrated and purified by column chromatography (0-60% EtOAc/hexanes). The two main products did not separate. To the mixture of products was added ethanol (1 mL) and iron (III) chloride (0.038 g, 0.231 mmol) in water (0.4 mL) acidified with 1 drop of 2 N HCl. Toluene (0.3 mL) was added to improve solubility. The reaction was stirred at room temperature for 15 h. The reaction was partitioned between water and ethyl acetate. The organic layer was concentrated and purified by column chromatography (0-100% EtOAc/hexanes). Product containing fractions were collected and concentrated giving 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I) (6.5 mg, 11.2% yield).

Method C: To a slurry of 4-((6-(2-(2,4-difluorophenyl)-1,1-difluoro-3-hydrazino-2-hydroxypropyl)pyridin-3-yl)oxy)benzonitrile (II) (0.5 g, 1.156 mmol) in ethyl acetate (4.63 mL) was added formaldehyde (37% in water, 0.086 mL, 1.156 mmol) and the mixture was stirred at RT for 30 min. Sodium thiocyanate (0.094 g, 1.156 mmol), sodium hydrogen sulfate (0.278 g, 2.313 mmol), and water (0.5 mL) was added and the reaction was stirred at RT for 19 h. Iron (III) chloride (10% w/v in water, 3.73 mL, 2.313 mmol) was added and the reaction stirred at RT for 2 h. The reaction was partitioned between ethyl acetate and water. The organic layer was washed with saturated sodium thiosulfate. 96.2 mg naphthalene was added to the organic layer for quantitation of 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I) by HPLC internal standard assay (250 mg, 43% in pot yield).

The process described in Example 1 may be conducted at temperatures ranging from about 0° C. to about 100° C., or from about 10° C. to about 50° C.

Thiocyanate reagents for use in this process may include potassium thiocyanate, sodium thiocyanate, or ammonium thiocyanate.

Formaldehyde or other sources of formaldehyde that may be used in this process include, but are not limited to, paraformaldehyde, gaseous formaldehyde, or formalin solution (i.e., an aqueous solution of formaldehyde).

Acids for use in this process may include acetic acid, sodium hydrogen sulfate, and potassium hydrogen sulfate.

Oxidants for use in this process may include iron (III) compounds such as, for example, iron (III) chloride, iron (III) bromide, and iron (III) acetylacetonate, sodium hypochlorite such as, for example, bleach, manganese dioxide (MnO₂), and hydrogen peroxide.

Solvents for use in this process may include ethyl acetate, tetrahydrofuran (THF), 2-MeTHF, acetonitrile (MeCN), N,N-dimethylformamide (DMF), N-methyl-2-pyrrolidone (NMP), methyl t-butyl ether (MTBE), ethanol, and mixtures thereof.

Compound IIa is formed as an intermediate in this process and is not isolated, but converted to compound I by treatment with the oxidant.

4-((6-(2-(2,4-Difluorophenyl)-1,1-difluoro-3-hydrazino-2-hydroxypropyl)pyridin-3-yl)oxy)benzonitrile (II) may be prepared from 4-((6-(2-(2,4-difluorophenyl)oxiran-2-yl)-difluoromethyl)pyridin-3-yl)oxy)benzonitrile (III) as shown in Example 2.

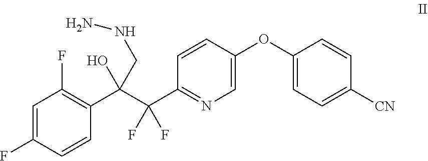 [SMILES: N#Cc1ccc(Oc2ccc(C(F)(F)C(O)(CNN)c3ccc(F)cc3F)nc2)cc1]

### Example 2

### Preparation of 4-((6-(2-(2,4-difluorophenyl)-1,1-difluoro-3-hydrazino-2-hydroxypropyl)pyridin-3-yl)oxy)benzonitrile (II)

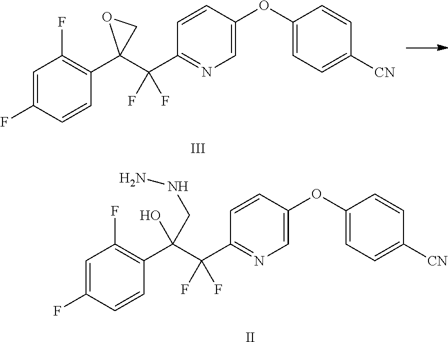

To a slurry of 4-((6-((2-(2,4-difluorophenyl)oxiran-2-yl)difluoromethyl)pyridin-3-yl)oxy)benzonitrile (III) (5 g, 12.49 mmol) in ethanol (50.0 mL) was added anhydrous hydrazine (1.0 mL, 31.2 mmol, 2.5 equiv.) and the reaction was heated at 60° C. for 4 h. The reaction was allowed to cool to room temperature overnight to give a white precipitate. The precipitate was isolated by filtration, and washed with ethanol (15 mL) and MTBE (15 mL). The solid was dried under vacuum giving 4-((6-(2-(2,4-difluorophenyl)-1,1-difluoro-3-hydrazino-2-hydroxypropyl)pyridin-3-yl)oxy)benzonitrile (II) (4.4 g, ˜85% purity, 69.3% corrected yield) as an off-white solid. ¹H NMR (400 MHz, CDCl₃) δ 8.38 (d, J=2.6 Hz, 1H), 7.70-7.65 (m, 2H), 7.64-7.55 (m, 1H), 7.45 (d, J=8.6 Hz, 1H), 7.36 (dd, J=8.7, 2.7 Hz, 1H), 7.08-7.02 (m, 2H), 6.85-6.71 (m, 2H), 3.76 (d, J=13.4 Hz, 1H), 3.62 (dd, J=13.4, 2.3 Hz, 1H). ¹⁹F NMR (376 MHz, CDCl₃) δ −105.40 (ddd, J=21.5, 16.2, 8.8 Hz), −109.57 (d, J=21.5 Hz), −109.77 (d, J=16.1 Hz), −110.58 (d, J=8.8 Hz). ESI MS m/z 433.1 [(M+H)⁺].

Solvents for use in this process step may include protic solvents selected from the group including methanol, ethanol, 1-propanol, and 2-propanol, and aprotic solvents selected from THF, acetonitrile, DMSO, and NMP.

This process step may be conducted at temperatures ranging from about 25° C. to about 100° C., or from about 40° C. to about 80° C.

What is claimed is:

1. A method of making a compound of Formula I comprising: 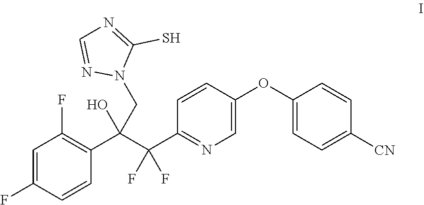 [SMILES: N#Cc1ccc(Oc2ccc(C(F)(F)C(O)(Cn3ncnc3S)c3ccc(F)cc3F)nc2)cc1]
contacting a compound of Formula II 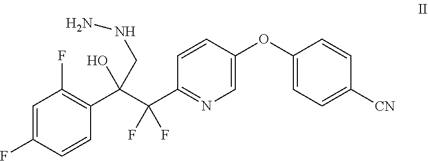 [SMILES: N#Cc1ccc(Oc2ccc(C(F)(F)C(O)(CNN)c3ccc(F)cc3F)nc2)cc1]
with formaldehyde or a source of formaldehyde, a thiocyanate salt and an oxidant.

2. The method of claim 1, wherein the thiocyanate salt is selected from the group including potassium thiocyanate, sodium thiocyanate, and ammonium thiocyanate.

3. The method of claim 1, wherein the oxidant comprises an iron (III) compound, sodium hypochlorite, manganese dioxide, or hydrogen peroxide.

4. The method of claim 3, wherein the iron (III) compound is iron (III) chloride, iron (III) bromide, or iron (III) acetylacetonate.

5. The method of claim 3, wherein the iron (III) compound is iron (III) chloride.

6. The method of claim 1 further comprising a solvent selected from the group including ethyl acetate, tetrahydrofuran, 2-MeTHF, acetonitrile, N,N-dimethylformamide, N-methyl-2-pyrrolidone, methyl t-butyl ether, ethanol, and mixtures thereof.

7. The method of claim 1 further comprising an acid selected from the group including acetic acid, sodium hydrogen sulfate, and potassium hydrogen sulfate.

8. The method of claim 1 wherein the contacting is carried out between about 0° C. and about 100° C.

9. The method of claim 1 wherein the contacting is carried out between about 10° C. and about 50° C.

10. The method of claim 1, further comprising the step of contacting a compound of Formula III 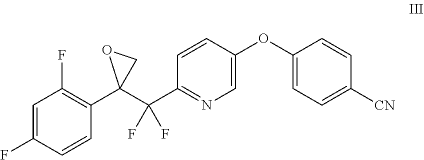 [SMILES: N#Cc1ccc(Oc2ccc(C(F)(F)C3(c4ccc(F)cc4F)CO3)nc2)cc1]
with hydrazine to produce the compound of Formula II.

11. The method of claim 10 further comprising a solvent selected from methanol, ethanol, 1-propanol, 2-propanol, THF, acetonitrile, DMSO, NMP, and mixtures thereof.

12. The method of claim 10 wherein the contacting is carried out between about 25° C. and about 100° C.

13. The method of claim 10 wherein the contacting is carried out between about 40° C. and about 80° C.

14. A compound selected from the group consisting of: 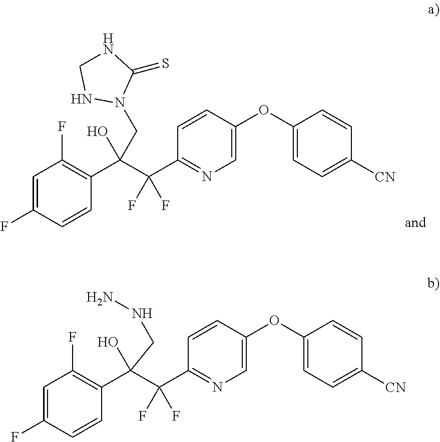 [SMILES: N#Cc1ccc(Oc2ccc(C(F)(F)C(O)(CN3NCNC3=S)c3ccc(F)cc3F)nc2)cc1 ; N#Cc1ccc(Oc2ccc(C(F)(F)C(O)(CNN)c3ccc(F)cc3F)nc2)cc1]
